(19) United States
(12) Patent Application Publication
(10) Pub. No.: US 2012/0029307 A1
(43) Pub. Date: Feb. 2, 2012

(54) VITAL-SIGNS MONITOR WITH SPACED ELECTRODES

(75) Inventors: Pierre PAQUET, Quebec (CA); Binta DIALLO, Quebec (CA)

(73) Assignee: CareFusion 303, Inc., San Diego, CA (US)

(21) Appl. No.: 12/844,769

(22) Filed: Jul. 27, 2010

(51) Int. Cl. A61B 5/00

(52) U.S. Cl. 600/301

(57) ABSTRACT

A vital-signs monitoring patch is disclosed. The patch includes at least two electrodes and a circuit assembly that periodically takes at least one measurement from the electrodes. The patch is a unitized device that contains the circuit assembly with the electrodes on the underside of the patch. The patch is attached to a patient with the electrodes in electrical contact with the patient's skin. The segments of the patch that connect the electrodes to the circuit assembly are flexible which reduces the noise induced in the measurement by stress on the contact between the electrodes and the patient.

### CROSS-REFERENCES TO RELATED APPLICATIONS

The following applications disclose certain common subject matter with the present application: A Vital-Signs Monitor with Encapsulation Arrangement, docket number 080624-0612; A Vital-Signs Patch Having a Strain Relief, docket number 080624-0624; A Temperature Probe Suitable for Axillary Reading, docket number 080624-0625; System and Method for Monitoring Body Temperature of a Person, docket number 080624-0626; System and Method for Saving Battery Power in a Vital Signs Monitor, docket number 080624-0628; A System and Method for Storing and Forwarding Data from a Vital-Signs Monitor, docket number 080624-0627; System and Method for Saving Battery Power in a Vital Signs Monitor, docket number 080624-0628; A System and Method for Conserving Battery Power in a Patient Monitoring System, docket number 080624-0629; A System and Method for Saving Battery Power in a Patient Monitoring System, docket number 080624-0630; A System And Method for Tracking Vital-Signs Monitor Patches, Docket Number 080624-0631; A System And Method for Reducing False Alarms Associated with Vital-Signs Monitoring, docket number 080624-0632; A System And Method for Location Tracking of Patients in a Vital-Signs Monitoring System, docket number 080624-0633; A System And Method for Reducing False Alarms Based on Motion and Location Sensing, docket number 080624-0634; all of the listed applications filed on ______.

### BACKGROUND

1. Field

The present disclosure generally relates to systems and methods of physiological monitoring, and, in particular, relates to monitoring of vital signs of patients.

2. Description of the Related Art

Some of the most basic indicators of a person's health are those physiological measurements that reflect basic body functions and are commonly referred to as a person's “vital signs.” The four measurements commonly considered to be vital signs are body temperature, pulse rate, blood pressure, and respiratory rate. Some clinicians consider oxygen saturation (S₀₂) to be a “fifth vital sign” particularly for pediatric or geriatric cases. Some or all of these measurements may be performed routinely upon a patient when they arrive at a healthcare facility, whether it is a routine visit to their doctor or arrival at an Emergency Room (ER).

Vital signs are frequently taken by a nurse using basic tools including a thermometer to measure body temperature, a sphygmomanometer to measure blood pressure, and a watch to count the number of breaths or the number of heart beats in a defined period of time which is then converted to a “per minute” rate. If a patient's pulse is weak, it may not be possible to detect a pulse by hand and the nurse may use a stethoscope to amplify the sound of the patient's heart beat so that she can count the beats. Oxygen saturation of the blood is most easily measured with a pulse oximeter.

When a patient is admitted to a hospital, it is common for vital signs to be measured and recorded at regular intervals during the patient's stay to monitor their condition. A typical interval is 4 hours, which leads to the undesirable requirement for a nurse to awaken a patient in the middle of the night to take vital sign measurements.

When a patient is admitted to an ER, it is common for a nurse to do a “triage” assessment of the patient's condition that will determine how quickly the patient receives treatment. During busy times in an ER, a patient who does not appear to have a life-threatening injury may wait for hours until more-serious cases have been treated. While the patient may be reassessed at intervals while awaiting treatment, the patient may not be under observation between these reassessments.

Measuring certain vital signs is normally intrusive at best and difficult to do on a continuous basis. Measurement of body temperature, for example, is commonly done by placing an oral thermometer under the tongue or placing an infrared thermometer in the ear canal such that the tympanic membrane, which shared blood circulation with the brain, is in the sensor's field of view. Another method of taking a body temperature is by placing a thermometer under the arm, referred to as an “axillary” measurement as axilla is the Latin word for armpit. Skin temperature can be measured using a stick-on strip that may contain panels that change color to indicate the temperature of the skin below the strip.

Measurement of respiration is easy for a nurse to do, but relatively complicated for equipment to achieve. A method of automatically measuring respiration is to encircle the upper torso with a flexible band that can detect the physical expansion of the rib cage when a patient inhales. An alternate technique is to measure a high-frequency electrical impedance between two electrodes placed on the torso and detect the change in impedance created when the lungs fill with air. The electrodes are typically placed on opposite sides of one or both lungs, resulting in placement on the front and back or on the left and right sides of the torso, commonly done with adhesive electrodes connected by wires or by using a torso band with multiple electrodes in the strap.

Measurement of pulse is also relatively easy for a nurse to do and intrusive for equipment to achieve. A common automatic method of measuring a pulse is to use an electrocardiograph (ECG or EKG) to detect the electrical activity of the heart. An EKG machine may use 12 electrodes placed at defined points on the body to detect various signals associated with the heart function. Another common piece of equipment is simply called a “heart rate monitor.” Widely sold for use in exercise and training, heart rate monitors commonly consist of a torso band, in which are embedded two electrodes held against the skin and a small electronics package. Such heart rate monitors can communicate wirelessly to other equipment such as a small device that is worn like a wristwatch and that can transfer data wirelessly to a PC.

Nurses are expected to provide complete care to an assigned number of patients. The workload of a typical nurse is increasing, driven by a combination of a continuing shortage of nurses, an increase in the number of formal procedures that must be followed, and an expectation of increased documentation. Replacing the manual measurement and logging of vital signs with a system that measures and records vital signs would enable a nurse to spend more time on other activities and avoid the potential for error that is inherent in any manual procedure.

### SUMMARY

For some or all of the reasons listed above, there is a need to be able to continuously monitor patients in different settings. In addition, it is desirable for this monitoring to be done with limited interference with a patient's mobility or interfering with their other activities.

Embodiments of the patient monitoring system disclosed herein measure certain vital signs of a patient, which include respiratory rate, pulse rate, blood pressure, body temperature, and, in some cases, oxygen saturation (S_{O2}), on a regular basis and compare these measurements to defined limits.

In certain aspects of the present disclosure, a vital-signs monitor patch is disclosed. The monitor includes at least two electrodes, a transmitter, a memory, and a processor. The monitor patch can be attached to a patient with the electrodes in electrical contact with the patient's skin with a separation of the electrodes of less than 20 centimeters. The processor periodically takes measurements from the electrodes, converts the measurement to vital sign signals, and causes the transmitter to transmit the vital sign signals.

In certain aspects of the present disclosure, a method of measuring respiration rate is disclosed. The method comprises placing two electrodes in electrical contact with a patient's skin, wherein the electrodes are separated by less than 20 centimeters, providing an electrical signal across the electrodes with a voltage oscillating at a frequency of 20-100 kHz across the electrodes with the current controlled to be constant at a value less than or equal to 10 microamperes where the combination of frequency, voltage, current, and electrode separation has been chosen to provide a voltage differential between the electrodes that is detectably modulated by the expansion and contraction of the patient's lungs while reducing the noise created by other physical activity of the patient, measuring the voltage differential between the two electrodes, analyzing the measurements to determine a respiration rate, and reporting the respiration rate.

In certain aspects of the present disclosure, a method of measuring cardiac pulse rate is disclosed. The method includes placing two electrodes in electrical contact with a patient's skin with a separation of less than 20 centimeters, measuring the voltage differential between the two electrodes, analyzing the measurements to determine a pulse rate, and reporting the pulse rate.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 is a diagram illustrating an exemplary embodiment of a patient monitoring system according to certain aspects of the present disclosure.

FIG. 2A is a perspective view of the vital-signs monitor patch of FIG. 1 according to certain aspects of the present disclosure.

FIG. 2B is a cross-section of the vital-signs monitor patch of FIG. 1 according to certain aspects of the present disclosure.

FIG. 2C is a functional block diagram illustrating exemplary electronic and sensor components of the vital-signs monitor patch of FIG. 1 according to certain aspects of the present disclosure.

FIG. 3 is a cross-section of the electrodes in place on the skin of a patient according to certain aspects of the present disclosure.

FIG. 4 is a partial schematic of the sensor interface of FIG. 2C according to certain aspects of the present disclosure.

FIG. 5 is a representative waveform of the output from the sensor interface of FIG. 2C according to certain aspects of the present disclosure.

FIG. 6 is a cross-section of an exemplary embodiment of the vital-signs monitor patch according to certain aspects of the present disclosure.

### DETAILED DESCRIPTION

Periodic monitoring of patients in a hospital is desirable at least to ensure that patients do not suffer an un-noticed sudden deterioration in their condition or a secondary injury during their stay in the hospital. It is impractical to provide continuous monitoring by a clinician and cumbersome to connect sensors to a patient, which are then connected to a fixed monitoring instrument by wires. Furthermore, systems that sound an alarm when the measured value exceeds a threshold value may sound alarms so often and in situations that are not truly serious that such alarms are ignored by clinicians.

Measuring vital signs is difficult to do on a continuous basis. Accurate measurement of cardiac pulse, for example, can be done using an electrocardiograph (ECG or EKG) to detect the electrical activity of the heart. An EKG machine may use up to 12 electrodes placed at various points on the body to detect various signals associated with the cardiac function. Another common piece of equipment is termed a “heart rate monitor.” Widely sold for use in exercise and physical training, heart rate monitors may comprise a torso band in which are embedded two electrodes held against the skin and a small electronics package. Such heart rate monitors can communicate wirelessly to other equipment such as a small device that is worn like a wristwatch and that can transfer data wirelessly to a personal computer (PC).

Monitoring of patients that is referred to as “continuous” is frequently periodic, in that measurements are taken at intervals. In many cases, the process to make a single measurement takes a certain amount of time, such that even back-to-back measurements produce values at an interval equal to the time that it takes to make the measurement. For the purpose of vital sign measurement, a sequence of repeated measurements can be considered to be “continuous” when the vital sign is not likely to change an amount that is of clinical significance within the interval between measurements. For example, a measurement of blood pressure every 10 minutes may be considered “continuous” if it is considered unlikely that a patient's blood pressure can change by a clinically significant amount within 10 minutes. The interval appropriate for measurements to be considered continuous may depend on a variety of factors including the type of injury or treatment and the patient's medical history. Compared to intervals of 4-8 hours for manual vital sign measurement in a hospital, measurement intervals of 30 minutes to several hours may still be considered “continuous.”

Certain exemplary embodiments of the present disclosure include a system that comprises a vital-signs monitor patch that is attached to the patient, and a bridge that communicates with monitor patches and links them to a central server that processes the data, where the server can send data and alarms to a hospital system according to algorithms and protocols defined by the hospital.

The construction of the vital-signs monitor patch is described according to certain aspects of the present disclosure. As the patch may be worn continuously for a period of time that may be several days, as is described in the following disclosure, it is desirable to encapsulate the components of the patch such that the patient can bathe or shower and engage in their normal activities without degradation of the patch function. An exemplary configuration of the construction of the patch to provide a hermetically sealed enclosure about the electronics is disclosed.

In the following detailed description, numerous specific details are set forth to provide a full understanding of the present disclosure. It will be apparent, however, to one ordinarily skilled in the art that embodiments of the present disclosure may be practiced without some of the specific details. In other instances, well-known structures and techniques have not been shown in detail so as not to obscure the disclosure.

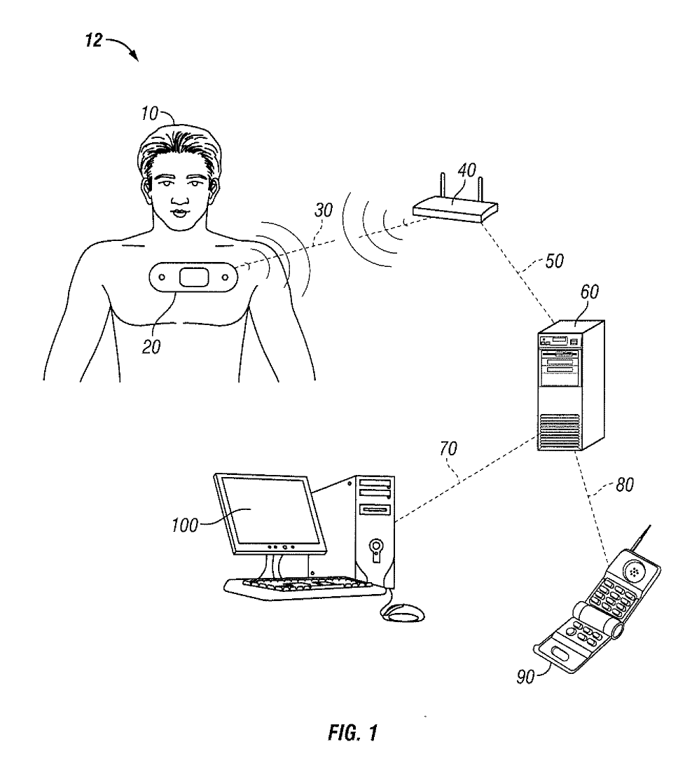

FIG. 1 discloses a vital sign monitoring system according to certain embodiments of the present disclosure. The vital sign monitoring system 12 includes vital-signs monitor patch 20, bridge 40, and surveillance server 60 that can send messages or interact with peripheral devices exemplified by mobile device 90 and workstation 100.

Monitor patch 20 resembles a large adhesive bandage and is applied to a patient 10 when in use. It is preferable to apply the monitor patch 20 to the upper chest of the patient 10 although other locations may be appropriate in some circumstances. Monitor patch 20 incorporates one or more electrodes (not shown) that are in contact with the skin of patient 10 to measure vital signs such as cardiac pulse rate and respiration rate. Monitor patch 20 also may include other sensors such as an accelerometer, temperature sensor, or oxygen saturation sensor to measure other characteristics associated with the patient. These other sensors may be internal to the monitor patch 20 or external sensors that are operably connected to the monitor patch 20 via a cable or wireless connection. Monitor patch 20 also includes a wireless transmitter that can both transmit and receive signals. This transmitter is preferably a short-range, low-power radio frequency (RF) device operating in one of the unlicensed radio bands. One band in the United States (US) is, for example, centered at 915 MHz and designated for industrial, scientific and medical (ISM) purposes. An example of an equivalent band in the European Union (EU) is centered at 868 MHz. Other frequencies of operation may be possible dependent upon the International Telecommunication Union (ITU), local regulations and interference from other wireless devices.

Surveillance server 60 may be a standard computer server connected to the hospital communication network and preferably located in the hospital data center or computer room, although other locations may be employed. The server 60 stores and processes signals related to the operation of the patient monitoring system 12 disclosed herein including the association of individual monitor patches 20 with patients 10 and measurement signals received from multiple monitor patches 20. Hence, although only a single patient 10 and monitor patch 20 are depicted in FIG. 1, the server 60 is able to monitor the monitor patches 20 for multiple patients 10.

Bridge 40 is a device that connects, or “bridges”, between monitor patch 20 and server 60. Bridge 40 communicates with monitor patch 20 over communication link 30 operating, in these exemplary embodiments, at approximately 915 MHz and at a power level that enables communication link 30 to function up to a distance of approximately 10 meters. It is preferable to place a bridge 40 in each room and at regular intervals along hallways of the healthcare facility where it is desired to provide the ability to communicate with monitor patches 20. Bridge 40 also is able to communicate with server 60 over network link 50 using any of a variety of computer communication systems including hardwired and wireless Ethernet using protocols such as 802.11a/b/g or 802.3af. As the communication protocols of communication link 30 and network link 50 may be very different, bridge 40 provides data buffering and protocol conversion to enable bidirectional signal transmission between monitor patch 20 and server 60.

While the embodiments illustrated by FIG. 1 employ a bridge 20 to provide communication link between the monitor patch 20 and the server 60, in certain alternative embodiments, the monitor patch 20 may engage in direct wireless communication with the server 60. In such alternative embodiments, the server 60 itself or a wireless modem connected to the server 60 may include a wireless communication system to receive data from the monitor patch 20.

In use, a monitor patch 20 is applied to a patient 10 by a clinician when it is desirable to continuously monitor basic vital signs of patient 10 while patient 10 is, in this embodiment, in a hospital. Monitor patch 20 is intended to remain attached to patient 10 for an extended period of time, for example, up to 5 days in certain embodiments, limited by the battery life of monitor patch 20. In some embodiments, monitor patch 20 is disposable when removed from patient 10.

Server 60 executes analytical protocols on the measurement data that it receives from monitor patch 20 and provides this information to clinicians through external workstations 100, preferably personal computers (PCs), laptops, or smart phones, over the hospital network 70. Server 60 may also send messages to mobile devices 90, such as cell phones or pagers, over a mobile device link 80 if a measurement signal exceeds specified parameters. Mobile device link 80 may include the hospital network 70 and internal or external wireless communication systems that are capable of sending messages that can be received by mobile devices 90.

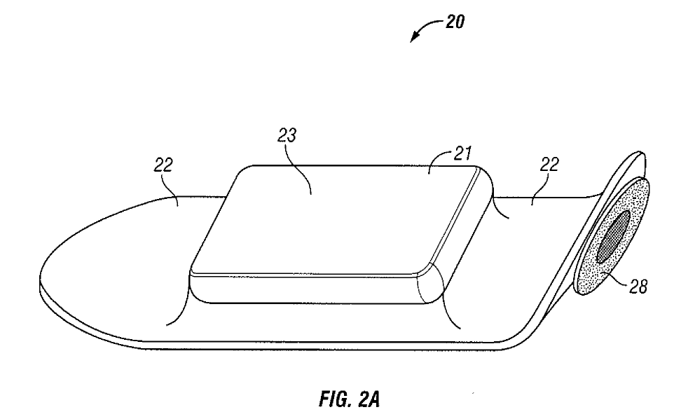

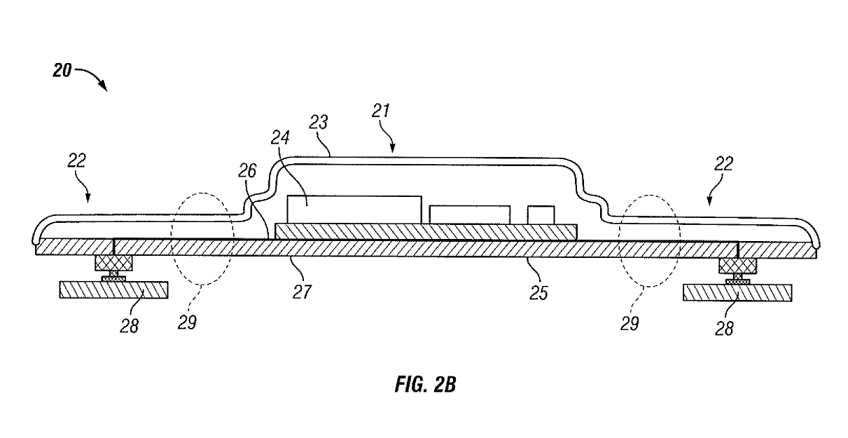

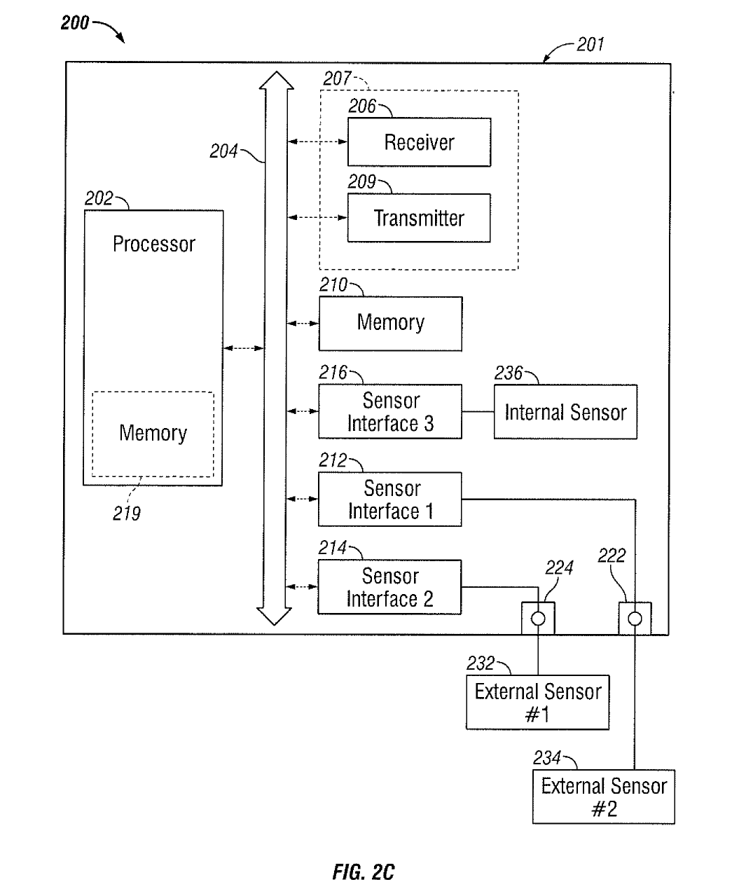

FIG. 2A is a perspective view of the vital-signs monitor patch 20 shown in FIG. 1 according to certain aspects of the present disclosure. In the illustrated embodiment, the monitor patch 20 includes component carrier 23 comprising a central segment 21 and side segments 22 on opposing sides of the central segment 21. In certain embodiments, the central segment 21 is substantially rigid and includes a circuit assembly (24, FIG. 2B) having electronic components and battery mounted to a rigid printed circuit board (PCB). The side segments 22 are flexible and include a flexible conductive circuit (26, FIG. 28) that connect the circuit assembly 24 to electrodes 28 disposed at each end of the monitor patch 20, with side segment 22 on the right shown as being bent upwards for purposes of illustration to make one of the electrodes 28 visible in this view.

FIG. 2B is a cross-sectional view of the vital-signs patch 20 shown in FIGS. 1 and 2A according to certain aspects of the present disclosure. The circuit assembly 24 and flexible conductive circuit 26 described above can be seen herein. The flexible conductive circuit 26 operably connects the circuit assembly 24 to the electrodes 28. Top and bottom layers 23 and 27 form a housing 25 that encapsulate circuit assembly 28 to provide a water and particulate barrier as well as mechanical protection. There are sealing areas on layers 23 and 27 that encircles circuit assembly 28 and is visible in the cross-section view of FIG. 2B as areas 29. Layers 23 and 27 are sealed to each other in this area to form a substantially hermetic seal. Within the context of certain aspects of the present disclosure, the term ‘hermetic’ implies that the rate of transmission of moisture through the seal is substantially the same as through the material of the layers that are sealed to each other, and further implies that the size of particulates that can pass through the seal are below the size that can have a significant effect on circuit assembly 24. Flexible conductive circuit 26 passes through portions of sealing areas 29 and the seal between layers 23 and 27 is maintained by sealing of layers 23 and 27 to flexible circuit assembly 28. The layers 23 and 27 are thin and flexible, as is the flexible conductive circuit 26, allowing the side segment 22 of the monitor patch 20 between the electrodes 28 and the circuit assembly 24 to bend as shown in FIG. 2A.

FIG. 2C is a functional block diagram 200 illustrating exemplary electronic and sensor components of the monitor patch 20 of FIG. 1 according to certain aspects of the present disclosure. The block diagram 200 shows a processing and sensor interface module 201 and external sensors 232, 234 connected to the module 201. In the illustrated example, the module 201 includes a processor 202, a wireless transceiver 207 having a receiver 206 and a transmitter 209, a memory 210, a first sensor interface 212, a second sensor interface 214, a third sensor interface 216, and an internal sensor 236 connected to the third sensor interface 216. The first and second sensor interfaces 212 and 214 are connected to the first and second external sensors 232, 234 via first and second connection ports 222, 224, respectively. In certain embodiments, some or all of the aforementioned components of the module 201 and other components are mounted on a PCB.

Each of the sensor interfaces 212, 214, 216 can include one or more electronic components that are configured to generate an excitation signal or provide DC power for the sensor that the interface is connected to and/or to condition and digitize a sensor signal from the sensor. For example, the sensor interface can include a signal generator for generating an excitation signal or a voltage regulator for providing power to the sensor. The sensor interface can further include an amplifier for amplifying a sensor signal from the sensor and an analog-to-digital converter for digitizing the amplified sensor signal. The sensor interface can further include a filter (e.g., a low-pass or bandpass filter) for filtering out spurious noises (e.g., a 60 Hz noise pickup).

The processor 202 is configured to send and receive data (e.g., digitized signal or control data) to and from the sensor interfaces 212, 214, 216 via a bus 204, which can be one or more wire traces on the PCB. Although a bus communication topology is used in this embodiment, some or all communication between discrete components can also be implemented as direct links without departing from the scope of the present disclosure. For example, the processor 202 may send data representative of an excitation signal to the sensor excitation signal generator inside the sensor interface and receive data representative of the sensor signal from the sensor interface, over either a bus or direct data links between processor 202 and each of sensor interface 212, 214, and 216.

The processor 202 is also capable of communication with the receiver 206 and the transmitter 209 of the wireless transceiver 207 via the bus 204. For example, the processor 202 using the transmitter and receiver 209, 206 can transmit and receive data to and from the bridge 40. In certain embodiments, the transmitter 209 includes one or more of a RF signal generator (e.g., an oscillator), a modulator (a mixer), and a transmitting antenna; and the receiver 206 includes a demodulator (a mixer) and a receiving antenna which may or may not be the same as the transmitting antenna. In some embodiments, the transmitter 209 may include a digital-to-analog converter configured to receive data from the processor 202 and to generate a base signal; and/or the receiver 206 may include an analog-to-digital converter configured to digitize a demodulated base signal and output a stream of digitized data to the processor 202. In other embodiments, the radio may comprise a direct sequence radio, a software-defined radio, or an impulse spread spectrum radio.

The processor 202 may include a general-purpose processor or a specific-purpose processor for executing instructions and may further include a memory 219, such as a volatile or non-volatile memory, for storing data and/or instructions for software programs. The instructions, which may be stored in a memory 219 and/or 210, may be executed by the processor 202 to control and manage the wireless transceiver 207, the sensor interfaces 212, 214, 216, as well as provide other communication and processing functions.

The processor 202 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable device or a combination of devices that can perform calculations or other manipulations of information.

Information, such as program instructions, data representative of sensor readings, preset alarm conditions, threshold limits, may be stored in a computer or processor readable medium such as a memory internal to the processor 202 (e.g., the memory 219) or a memory external to the processor 202 (e.g., the memory 210), such as a Random Access Memory (RAM), a flash memory, a Read Only Memory (ROM), a Programmable Read-Only Memory (PROM), an Erasable PROM (EPROM), registers, a hard disk, a removable disk, or any other suitable storage device.

In certain embodiments, the internal sensor 236 can be one or more sensors configured to measure certain properties of the processing and sensor interface module 201, such as a board temperature sensor thermally coupled to a PCB. In other embodiments, the internal sensor 236 can be one or more sensors configured to measure certain properties of the patient 10, such as a motion sensor (e.g., an accelerometer) for measuring the patient's motion or position with respect to gravity.

The external sensors 232, 234 can include sensors and sensing arrangements that are configured to produce a signal representative of one or more vital signs of the patient to which the monitor patch 20 is attached. For example, the first external sensor 232 can be a set of sensing electrodes that are affixed to an exterior surface of the monitor patch 20 and configured to be in contact with the patient for measuring the patient's respiratory rate, and the second external sensor 234 can include a temperature sensing element (e.g., a thermocouple or a thermistor or resistive thermal device (RID)) affixed, either directly or via an interposing layer, to skin of the patient 10 for measuring the patient's body temperature. In other embodiments, one or more of the external sensors 232, 234 or one or more additional external sensors can measure other vital signs of the patient, such as blood pressure, pulse rate, or oxygen saturation.

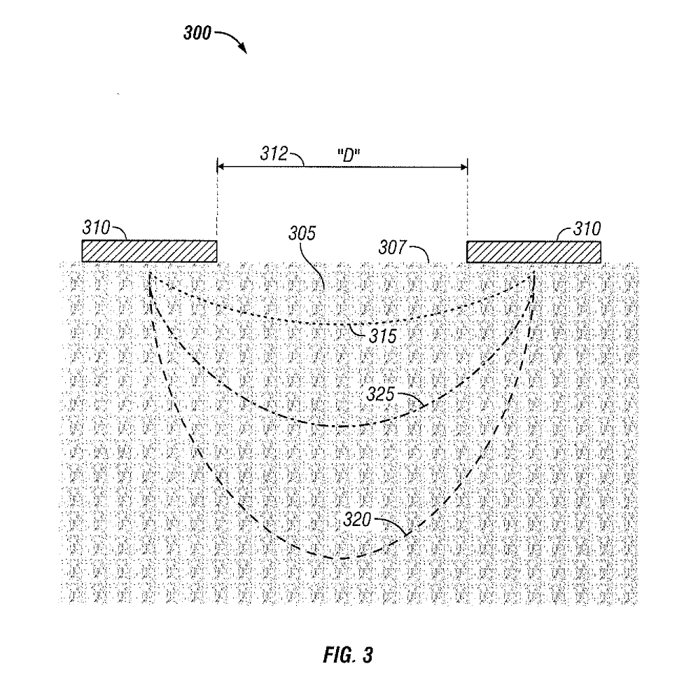

FIG. 3 is a cross-section 300 of the sensing electrodes placed on a patient's body. In this implementation, vital-signs monitor patch 20 comprises two electrodes 310 which are shown in the figure with the rest of monitor patch 20 omitted for clarity. Electrodes 310 are placed on the skin 307 of a patient's body 305. The electrodes are separated by distance 312 designated as “D”. The electrodes 310 are in electrical contact with skin 307 and connected to the rest of monitor patch 20 according to block diagram 200 of FIG. 2. A liquid or gel, not shown in this figure, may be applied between electrodes 310 and skin 307 to improve conductivity.

When a high-frequency alternating current (AC) voltage is applied across two electrodes that are in contact with the surface of a material that has a significant depth, the effective path of the conduction from one electrode to another is dependent upon the frequency of the applied voltage. Drive frequencies of 20-100 kHz have been found to penetrate far enough into the body, depending on the separation of the electrodes, to detect expansion and contraction of the lungs. In this example, a drive signal in the range of 25-40 kHz is applied across the two electrodes 310 and the current approximately follows the conduction path 325. At a drive frequency near 100 kHz, the current would not penetrate as deep into body 305 and would approximately follow path 315 which would produce a reduced signal. At drive frequencies near 20 kHz, the current would tend to penetrate more deeper into body 305 and would approximately follow path 320 but the higher resistance of the longer path 320 would increase the noise in the measurement and make it impossible to make a measurement at safe levels of drive voltage. Selection of the drive frequency is a balance of keeping the applied voltage and the current to safe levels while selecting a conduction path that is deep enough to detect the physiological parameter of interest.

To measure the respiration rate of a patient, one method of measurement, for example, is to select electrode 310 locations and a drive frequency such that conduction path 325 passes through at least a portion of the lungs so that the signal conducted along the conduction path 325 is modulated by the expansion of the lungs with air during inhalation and the collapse of the lungs during exhalation. An existing method is to place two electrodes on opposite sides of the lungs, such as one electrode on the front of the body and the second electrode on the back. The electrodes are either attached with adhesive and connected with wires to a measurement device, or embedded in a strap that is worn around the chest. In both cases, there are wires or straps around or across a large part of the body, obstructing access by the clinician and being uncomfortable to the patient. In the exemplary embodiment shown in FIG. 3, however, the electrodes 310 are placed in close proximity to each other, separated by a distance “D” 312 of less than 20 centimeters and preferably under 11 centimeters, and over the lungs, preferably on the upper chest area near the left-to-right center of the chest, and the drive signal is in the range of 20-100 kHz, preferably 32 kHz. As the separation of the electrodes 310 is reduced, the modulation of the signal by the expansion and contraction of the lungs is reduced while, at the same time, the noise artifacts in the signal that are created by movement of the patient are also reduced. Proper circuit design using precision components and principles of low-noise electronic design, known to those of ordinary skill in the art, enable the separation of electrodes 310 to be less than 20 centimeters. As the overall size of the patch 20 is a function of the separation of the electrodes 310, reducing the separation results in a smaller and less-intrusive patch 20. In the configuration of FIG. 3, placement of the electrodes 310 over the lungs, preferably near the center of the patient's upper chest, provides sufficient modulation of the drive signal by the expansion and collapse of the lungs to enable identification of each breath of the patient. Other locations for electrodes 310, such as on the side of the upper body or on the back of patient 10, provide similar proximity to the lungs and may be suitable for placement of a respiration sensor of the type claimed herein.

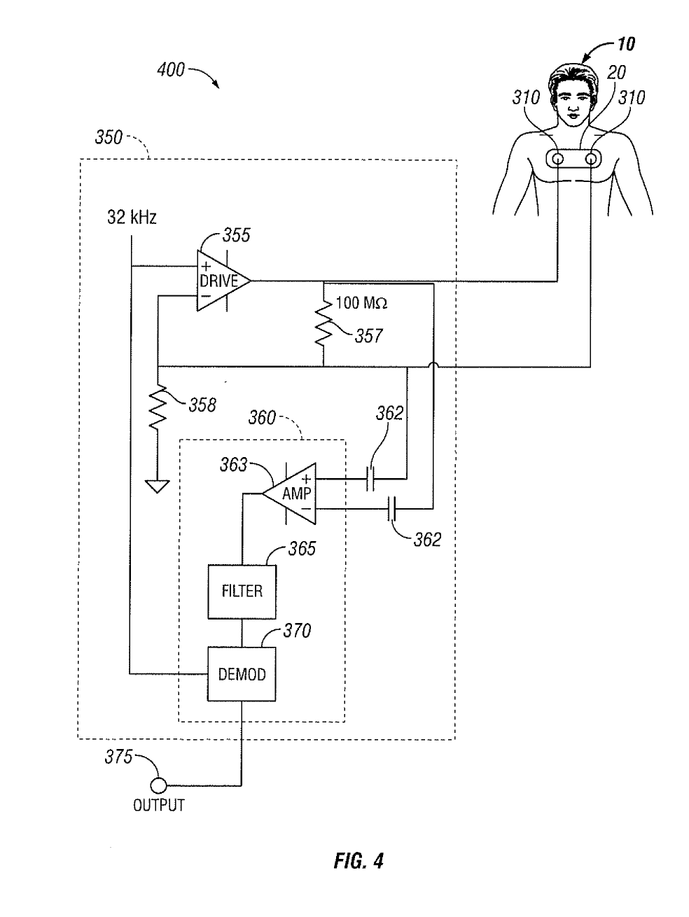

FIG. 4 is a simplified diagram of sensor interface 350 which is an exemplary embodiment of interface block 212 of FIG. 2. In this example, sensor 20 is attached to the upper chest of patient 10 with two electrodes 310 in contact with the skin of patient 10. Interface 350 includes a drive element 355 and a sense element 360, commonly connected as shown to electrodes 310. Drive 355 has an input that is the carrier frequency, and which is 32 kHz in this example. Other drive frequencies in the range of 20-100 kHz may be utilized, taking into consideration the tradeoff between signal strength and noise created by the different depths of conduction path 325 as discussed above. Drive 355 incorporates a feedback circuit (not shown) to control the current output of drive 355 to remain essentially constant at a safe level for conduction through the chest of a patient, preferably below 10 microamperes. The output of drive 355 is capacitively isolated by elements 364 from the patient to prevent any DC bias current from flowing through the electrodes. The voltage drop across the parallel network of resistor 357 and the impedance of the body of patient 10 is capacitively coupled through elements 362 to a differential amplifier 363 that is part of sense element 360. Sense element 360 also incorporates a filter element 365, preferably a bandpass filter, and a demodulation element 370 that removes the 32 kHz carrier and produces an output 375. Output 375 will be sampled and digitized, which may be accomplished as part of the sensor interface 212 or by processor 202, before further processing.

Cardiac pulse rate, commonly referred to simply as pulse, can be measured without a drive signal. The heart generates an electrical field as part of its normal beating where the field varies synchronously with the various steps in a heart's activity. An EKG machine uses a number of electrodes placed at specific points on the skin around the heart such that different parts of the heart's function can be detected by selecting various pairs of electrodes. However, simply detecting a repetitive signal that can be associated with pulse can be done with far fewer electrodes. The configuration of FIG. 3, for example, could detect the heart's electrical field if the electrodes 310 were suitably placed. A preferable location for measuring pulse is for the electrodes 310 to be placed on opposite sides of the heart such as, for example, with two electrodes 310 aligned vertically (i.e. both aligned to a horizontal line across the chest of a standing patient) and horizontally separated and centered on the patient (i.e. with one sensor a certain horizontal distance to the left of a vertical line down the center of the patient's chest and the other sensor a horizontal distance to the right of this vertical line). In this configuration, the two sensors are on opposite sides of the heart and commonly “above” the heart, rather than truly on opposite sides of the heart as is characteristic of the placement of EKG sensors. In this position, the two sensors can still detect a voltage differential created by the electrical activity of the heart. While the signal is reduced as the separation of the electrodes is reduced, noise is also reduced. Proper filtering and the use of low-noise components enables a high-quality measurement of a patient's pulse to be detected at a much smaller separation of electrodes than previously required in EKG measurements.

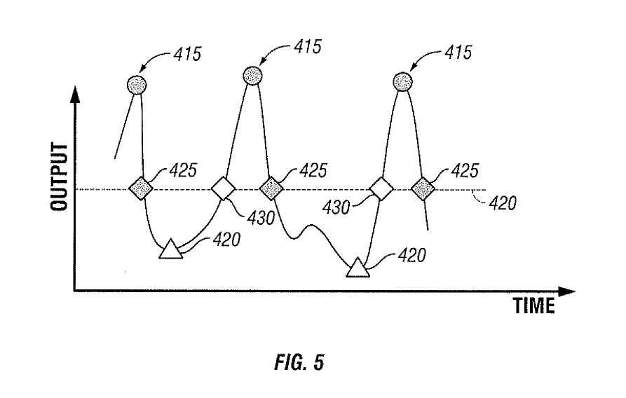

FIG. 5 is an exemplary plot 400 representative of respiration output 375 from FIG. 4 which represents the filtered, demodulated, and amplified voltage drop across electrodes 310. This plot is representative of the output that is obtained by either frequency demodulation or amplitude demodulation. The demodulation process can be configured to produce a reference level 405 where the signal fluctuates above and below this level. This is commonly considered to be the “zero” level for purposes of signal analysis. There are a plurality of analytical methods that can be used to identify specific features of a variable signal. For instance, a peak detector can detect the peaks 415 of each “wave”. In a different configuration, a peak detector could identify the valleys 420 of each wave. An alternate approach is to detect the “zero crossing”, i.e. where the signal value is approximately the same as the “zero” level, as the signal is decreasing, shown as points 425, or as the signal is increasing, shown as points 430. Any of these types of “markers” of a periodic signal can be used to identify each wave. The time at which the measurement of each marker point is made, therefore, becomes the time of that wave. In the example of measuring the expansion and collapse of a patient's lungs, where the analysis might use the peak voltage as the marker for an inhalation, the time of the peak value is the time of that breath. The rate of respiration can be calculated on a short-term basis using only the time interval since the last breath, where the rate (breaths per minute)=60 (seconds)/time interval (seconds). Alternately, the rate of respiration could be calculated on a long-term basis, for instance, based on the last 4 breaths, where the rate=(3 *60)/(total elapsed time from breath #1 to breath #4). Other methods of calculating a current rate may include weighted averaging, moving averages, threshold detection, or any other numerical analysis technique.

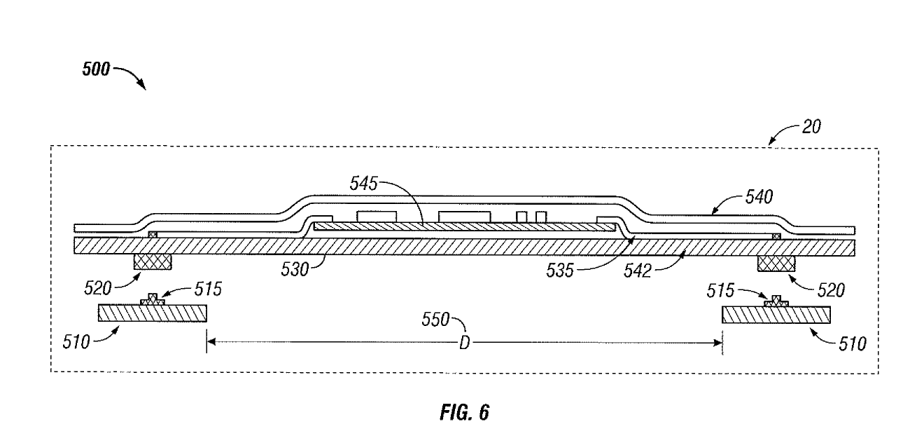

FIG. 6 is a cross-section 500 of an exemplary embodiment of the vital-signs monitor patch 20. In this embodiment, the electrodes 510 are of the type commonly used with EKG machines, wherein the electrode 510 comprises a flexible adhesive sheet with a central conductive area (not shown) and a snap fitting 515 that is electrically connected to the conductive area on side of the electrode 510 that touches the patient when in use. The main body of monitor patch 20 comprises a mating snap receptacle 520 at two locations to which electrodes 510, shown prior to their attachment to the main body of patch 20, can be attached. Electrodes 510 could alternately be implemented in other forms such as conductive areas on the underside of lower film 542 without departing from the scope of the claims. Monitor patch 20 is configured such that electrodes 510, when attached to receptacles 520, are separated by distance 550 designated “D”. These receptacles 520 are separately and electrically connected to the main circuit assembly 545 of monitor patch 20 through conductors 535, which may be discrete wires or conductive stripes printed onto lower film 542. A protective film 540 covers the upper surface of the main body of monitor patch 20. In this embodiment, monitor patch 20 is applied to the chest of a patient whereupon the electrodes 510 adhere to the patient's skin and provide the mechanical attachment.

Monitor patch 20 designs, according to the disclosed embodiments, employ electrodes that are spaced sufficiently far apart to obtain accurate measurements and close enough to provide a compact monitor patch 20. The unitized design of the disclosed embodiments of monitor patch 20 is easy to apply to a patient and the small size of monitor patch 20, together with the ability to transmit data wirelessly from monitor patch 20 to the rest of the patient monitoring system 12, does not interfere with the patient's movement about the hospital or hamper other activities of the patient.

It can be seen that the disclosed embodiments of the vital-signs monitor patch 20 provide a mobile solution to monitoring the vital signs of a patient. The design of the vital-signs monitor patch 20 frees nurses, or other caregivers, from the task of repetitively measuring the vital signs of their patients, allowing the caregivers to spend more time on other duties. The ability to continuously monitor a patient's vital signs using a monitor patch 20, together with the rest of the patient monitoring system 12, increases the ability of the nurse to respond quickly to a sudden change in a patient's condition, resulting in improved care for the patient.

It can be seen that the disclosed embodiments of the vital-signs monitor patch provide a mobile solution to monitoring the vital signs of a patient. The design of the vital-signs monitor patch frees nurses, or other caregivers, from the task of repetitively measuring the vital signs of their patients, allowing the caregivers to spend more time on other duties. The ability to continuously monitor a patient's vital signs using a monitor patch, together with the rest of the patient monitoring system, increases the ability of the nurse to respond quickly to a sudden change in a patient's condition, resulting in improved care for the patient. The compact design of the patch enabled by the reduced separation of the electrodes compared to existing products will increase the comfort of the patient and reduce limitations on activities of the patient, making it more likely that the patient will wear the patch, which improves the safety of the patient through continuous monitoring.

The previous description is provided to enable any person skilled in the art to practice the various aspects described herein. While the foregoing has described what are considered to be the best mode and/or other examples, it is understood that various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such an embodiment may refer to one or more embodiments and vice versa.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

What is claimed is:

1. A vital-signs monitor patch for attachment to the skin of a person, comprising:
a component carrier,
at least two electrodes coupled to the component carrier and positioned to contact the skin of the person to which the monitor patch is attached, the electrodes being separated from one another by less than 20 centimeters;
a transmitter coupled to the component carrier and configured to transmit vital-sign signals;
a memory coupled to the component carrier and configured to store executable instructions; and
a processor coupled to the component carrier and operably connected to the memory, transmitter, and electrodes, wherein the processor is configured to execute instructions to periodically take measurements from the electrodes, to convert the measurements to vital sign signals, and to cause the transmitter to transmit the vital sign signals.

2. The vital-signs patch of claim 1 wherein the measurements are related to at least one vital sign of the set of body temperature, cardiac pulse rate, respiration rate, blood pressure, and oxygen saturation.

3. The vital-signs patch of claim 2 wherein the patch takes measurements related to cardiac pulse rate and respiration rate.

4. The vital-signs patch of claim 3 wherein the patch also takes measurements related to body temperature.

5. The vital-signs patch of claim 4 wherein the patch also takes measurements related to blood pressure.

6. The vital-signs patch of claim 2 wherein the processor is configured to analyze one or more of the measurements to calculate the at least one vital sign, convert the calculated at least one vital sign to a data record, to store the data record in the memory, to retrieve at least a portion of the data record, and to configure the retrieved portion of the data record into the vital sign signal.

7. The vital-signs patch of claim 6 wherein the transmitter can also receive signals and the processor is configured to cause the transmitter to transmit the vital sign signal upon receipt of a signal received by the transmitter.

8. The vital-signs patch of claim 1 wherein the electrodes are connectors to which separate electrodes can be attached.

9. The vital-signs patch of claim 8 wherein the separate electrodes comprise a layer of adhesive on at least a portion of the electrodes.

10. The vital-signs patch of claim 1 wherein the patch is less than 25 centimeters in its longest dimension.

11. The vital-signs patch of claim 10 wherein the patch is less than 10 centimeters perpendicular to its longest dimension.

12. The vital-signs patch of claim 2 wherein the patch is configured to take a measure related to respiration rate by applying a signal oscillating at a frequency of 20-100 kHz across the electrodes, controlling the current to be constant at a value less than or equal to 10 microamperes, and measuring the voltage differential between the two electrodes.

13. The vital-signs patch of claim 2 wherein the patch is configured to take a measure related to cardiac pulse rate by measuring the voltage differential between the two electrodes.

14. A method of measuring respiration rate that comprises the steps of:
placing two electrodes in electrical contact with a patient's skin, wherein the electrodes are separated by less than 20 centimeters;
providing an electrical signal across the electrodes with a voltage oscillating at a frequency of 20-100 kHz and a current controlled to be constant at a value of less than or equal to 10 microamperes, wherein the combination of frequency, voltage, current, and electrode separation has been chosen to provide a voltage differential between the electrodes that is detectably modulated by the expansion and contraction of the patient's lungs while reducing the noise created by other physical activity of the patient;
measuring the voltage differential between the two electrodes;
analyzing the measurements to determine a respiration rate;
reporting the respiration rate.

15. The method of claim 14 wherein the electrodes are placed approximately in the center of the patient's upper chest and are horizontally separated.

16. The method of claim 14 wherein the step of analyzing the measurements comprises demodulation to derive an amplitude-modulated signal related to respiration.

17. A method of measuring cardiac pulse rate that comprises the steps of:
placing two electrodes in electrical contact with a patient's skin approximately, wherein the electrodes are separated by less than 20 centimeters;
measuring the voltage differential between the two electrodes;
analyzing the measurements to determine a pulse rate;
reporting the pulse rate.

18. The method of claim 17 wherein the electrodes are placed approximately in the center of the patient's upper chest and are horizontally separated.
